(19) United States
(12) Patent Application Publication
(10) Pub. No.: US 2006/0094805 A1
(43) Pub. Date: May 4, 2006

(54) Esters and partial esters of polyvaleny alcohols

(76) Inventor: Franz-Leo Heinrichs, Sulzbach (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/529,827

(22) PCT Filed: Sep. 22, 2003
(86) PCT No.: PCT/EP03/10502

§ 371 Date: Mar. 30, 2005

(30) Foreign Application Priority Data
Sep. 30, 2002 (DE) 102 45 623.2

(51) Int. Cl. C07C 59/185; C07C 69/34; C08K 5/09

(52) U.S. Cl. 524/284; 560/199; 554/115

(57) ABSTRACT

Esters or partial esters comprising at least one carboxylic acid radical and/or a radical of a carboxylic acid mixture having from 24 to 34 carbon atoms (component B) and contain at least one carboxylic acid radical and/or a radical of a carboxylic acid mixture having from 8 to 22 carbon atoms (component C). The esters and partial esters of the present invention find particular application as a processing adjunct for plastics, a dispersing agent for pigments or for the production of micronised products.

The invention relates to esters and partial esters of polyhydric alcohols and carboxylic acids and their use.

Esters of polyols and of long-chain carboxylic acids, in particular fatty acids, have been known for a long time and are widely used in industry. Examples of these are natural and synthetic glycerol esters of fatty acids, pentaerythritol tetrastearate, diglycerol stearates, oleates, pentaerythritol montanate, trimethylolpropane montanate, and many other compounds.

Many of these products are used in plastics processing, and sometimes products based on fatty acids are used alongside products based on montan wax acid, because their performance characteristics are complementary.

The fatty acids are single-chain-length products or products used with a chain length distribution, the chain lengths being C₈-C₁₈, and in exceptional cases saturated C₂₂ and unsaturated C₂₂ are also available.

The very important product montan wax acid is a mixture of long-chain carboxylic acid having from 24 to 34 carbon atoms, produced during the refining of montan wax.

The chain length distribution of the acids (i.e. fatty acids and montan wax acid) cause the derivatives of these acids to behave differently in plastics. The highly polar fatty acid derivatives have better compatibility in polar plastics and tend to act internally, while montan wax acid derivatives with their longer alkyl chains are less polar than fatty acid derivatives and tend to act externally in polar plastics. Both modes of action are desired and required for processing.

Lower molecular weight makes fatty derivatives more polar than montan derivatives. “Internal” and “external action” are terms especially used in the processing of PVC or of other plastics, relating to the site of action of lubricants. Internal agents act within the melt or in the case of PVC within the polymer grains, while external lubricants act at the melt/mold phase boundary and then also act as release agents. Non-polar lubricants act externally in polar plastics, while polar lubricants act externally in non-polar plastics, and vice versa.

Montan wax derivatives generally exhibit marked intrinsic color, and this generally prevents their use in transparent applications. Fatty acid esters have only slight intrinsic color, but, because their chain length is relatively short, are often too polar and too mobile or too volatile when subjected to high temperature.

There has therefore long been a desire for products with montan wax properties and little intrinsic color, or for products which have as little intrinsic color as fatty derivatives and have the properties of montan wax derivatives.

Surprisingly, it has now been found that esters whose molecule simultaneously contains both types of carboxylic acid (fatty acid and montan wax acid) exhibit the abovementioned desired properties.

The invention therefore provides esters and partial esters of the type mentioned at the outset, which contain at least one carboxylic acid radical and/or a radical of a carboxylic acid mixture having from 24 to 34 carbon atoms (component B) and contain at least one carboxylic acid radical and/or a radical of a carboxylic acid mixture having from 8 to 22 carbon atoms (component C).

The polyhydric alcohol preferably has from 3 to 12 hydroxy groups.

The polyhydric alcohol is preferably trimethylolpropane, trimethylolethane, pentaerythritol, glycerol, diglycerol, polyglycerol, sorbitol, dipentaerythritol, ditrimethylolpropane, and/or ethoxylated derivatives of these.

The polyhydric alcohol is particularly preferably trimethylolpropane, glycerol, and/or pentaerythritol.

The radical of the carboxylic acid mixture (component C) preferably contains from 8 to 18 carbon atoms.

The esters and partial esters also preferably contain at least one dicarboxylic acid radical and/or the radical of a dicarboxylic acid mixture (component D).

The dicarboxylic acid radical and/or the radical of the dicarboxylic acid mixtures preferably derive from compounds such as adipic acid, dodecanedioic acid, or dimer fatty acid.

The esters and partial esters of the present invention preferably have an acid number of from 5 to 25.

The esters and partial esters of the present invention preferably have a hydroxy number of from 5 to 400 (defined by DGF method M-IV-6 (57)).

The esters and partial esters of the present invention particularly preferably have a hydroxy number of from 5 to 150.

The invention also provides the use of ester or partial ester of the present invention as processing aid or in the processing of plastics.

The plastics are preferably thermoplastics.

The thermoplastics are particularly preferably polyvinyl chloride, polyester, polycarbonate, polyamide, or polypropylene.

The inventive esters and partial esters may be used as dispersing agents for pigments, and also as lubricants in the processing of plastics.

They may also be used for preparation of micronizates.

As is well known, the reaction of polyhydric alcohols with carboxylic acids to give esters always produces mixtures of esters having different degrees of esterification. If the reaction uses alcohols having three or more alcohol functions, e.g. trimethylolpropane, pentaerythritol, glycerol, sorbitol, ditrimethylolpropane, dipentaerythritol, diglycerol, or polyglycerol, it is possible, using a two-stage process and an acidic catalyst, first to prepare the partial esters of the high-molecular-weight acid (component B) and then to react all of the remaining, or some of the free, hydroxy groups with the more reactive low-molecular-weight acid (component C). This gives a polyol ester or, respectively, a partial ester in which carboxylic acids having from 8 to 22 and from 24 to 34 carbon atoms are incorporated in a specific manner.

Mixed esters of a polyol and of different carboxylic acid molecules, such as fatty acid or montan wax acid, which still contain free hydroxy functions can then be reacted with dibasic carboxylic acids (component D), such as adipic acid, dodecanedioic acid, or dimer fatty acid, to give oligomers.

The principle does not work with dihydric alcohols, because in that case the product is inevitably always diesters which contain only one type of carboxylic acid.

If an Sn-containing catalyst is used for the esterification process, the reaction can then also be carried out in a single-step process.

Examples of preparation of the inventive esters and partial esters.

### EXAMPLE 1

### Trimethylolpropane (TMP) Mixed Ester

Process:

Montan wax acid (1.5 mol) was melted at 100° C. and trimethylolpropane (1 mol) and 70% strength methanesulfonic acid (0.2% by weight) were added at that temperature and the mixture was then heated to 120° C. The mixture was inertized with N₂, and stirred for 3 hours while the water of reaction was removed by distillation, and then tallow fatty acid (1.2 mol) was added and the mixture was esterified until the acid number had fallen below 12. The catalyst was neutralized with 30% strength NaOH, and the final product was dried and filtered.

### EXAMPLE 2

### Glycerol Mixed Ester

Montan wax acid (1.1 mol) and tallow fatty acid (1.2 mol) were melted at 100° C., and glycerol (1 mol) and Fascat 2001 (0.1% by weight) were added at that temperature, and the mixture was then heated to 190° C. The mixture was inertized with N₂, and stirred while the water of reaction was removed by distillation, until the acid number had fallen below 15, then cooled to 120° C. and filtered.

### EXAMPLE 3

### Pentaerythritol Mixed Ester

Montan wax acid (1.5 mol) and tallow fatty acid (2 mol) were melted at 100° C., and pentaerythritol (1 mol) and Fascat 2001 (0.1% by weight) were added at that temperature, and the mixture was then heated to 190° C. The mixture was inertized with N₂, and stirred while the water of reaction was removed by distillation, until the acid number had fallen below 20, then cooled to 120° C. and filtered.

### EXAMPLE 4

### Pentaerythritol Complex Ester

Montan wax acid (1.1 mol) and tallow fatty acid (2 mol) were melted at 100° C., and pentaerythritol (1 mol) and Fascat 2001 (0.1% by weight) were added at that temperature, and the mixture was then heated to 190° C. The mixture was inertized with N₂, and stirred while the water-of reaction was removed by distillation, until the acid number had fallen below 40, the mixture then being treated with adipic acid and esterified until the AN had fallen below 20, then cooled to 120° C. and filtered.

TABLE 1
Properties of starting materials and products

| | AN | SN | FH | Dp | MV |
|---|---|---|---|---|---|
| Type | mg KOH | mg KOH | bar | ° C. | mPas |
| Trimethylolpropane | 11.8 | 145.1 | 237 | 62.9 | 27 |
| Glycerol | 13.12 | 156.8 | 340 | 71.9 | 26 |
| Pentaerythritol | 12 | 153 | 380 | 71.9 | 39 |
| Pentaerythritol | 17.5 | 188.6 | 280 | 69 | 42 |
| complex of Example 4 | | | | | |

AN = Acid number to DIN 53402
SN = Saponification number to DIN 53401
FH = Flow hardness
Dp = Drop point ° C. to DIN 51801/2
MV = Melt viscosity at 100° C. to DIN 51562

The products prepared by the processes described were tested in the processing of various plastics, and when compared with pure montan wax derivatives exhibited an improvement in color properties without any significant losses in performance.

Applications Testing

TABLE 2
Volatility

Volatility in %

| Type | 220° C. | 260° C. |
|---|---|---|
| Trimethylolpropane | 0.4 | 1.3 |
| Glycerol | 0.5 | 2 |
| Pentaerythritol | 0.5 | 2 |
| Pentaerythritol complex of Example 4 | 0.5 | 1.7 |
| Glycerol monostearate | 1.8 | 6.9 |
| Fatty acid complex ester* | 0.6 | 2.2 |
| Glycerol montanate | 0.4 | 1.2 |
| Pentaerythritol montanate | 0.5 | 1.3 |
| Montanic acid complex ester** | 0.4 | 1.2 |

* ®Loxiol G70S from Cognis
** ®Licolub WE 40 from Clariant GmbH

TABLE 3
Yellowness index (measurement of yellowing
of near-white materials to DIN 6167)

| | Yellowness index in scale units | | | |
|---|---|---|---|---|
| | (at test product content of) | | | |
| | 0.3% | | 0.5% | |
| | after | after | after | after |
| Ester type | 5 min | 15 min | 5 min | 15 min |
| Trimethylolpropane mixed ester | 2.9 | 4.1 | 3.4 | 7 |
| Glycerol mixed ester | | | | |
| Pentaerythritol mixed ester | | | | |
| Pentaerythritol complex ester | | | | |
| of Example 4 | | | | |
| TMP montanate | 2.9 | 7.2 | 4 | 8.2 |
| Fatty acid complex ester* | 2.9 | 7.2 | 3.8 | 7.8 |
| Glycerol montanate | 3.5 | 8.3 | 5.1 | 9 |
| Pentaerythritol montanate | 3.6 | 7.5 | 5.8 | 10 |
| Montanic acid complex ester** | 3.8 | 7.5 | 3.8 | 8.8 |

* ®Loxiol G70S from Cognis
** ®Licolub WE 40 from Clariant GmbH

The following mixing specification (in % by weight) was used for the measurements and test results in Table 3 to 5:

| | | |
|---|---|---|
| ®Vinnolit S 3160 | 100% | by weight |
| ®Irgastab 17 MOK | 1.5% | by weight |
| ®Paraloid K 120 N | 1% | by weight |
| ®Loxiol G 16 | 0.3% | by weight |

Test product (\hat{=} ester type) optionally 0.3 or 05.% by weight

TABLE 4
Transparency (to ASTM D1003)

| | Transparency in % | | | |
|---|---|---|---|---|
| | (at test product content of) | | | |
| | 0.3% | | 0.5% | |
| | after | after | after | after |
| Ester type | 5 min | 15 min | 5 min | 15 min |
| TMP mixed ester | 83 | 79 | 67 | 74 |
| Glycerol mixed ester | 85 | 83 | 77 | 80 |
| Pentaerythritol mixed ester | 79 | 80 | 68 | 70 |
| Pentaerythritol complex ester | 84 | 82 | 77 | 77 |
| of Example 4 | | | | |
| Glycerol monostearate | 78 | 80 | 66 | 73 |
| Fatty acid complex ester* | 86 | 82 | 83 | 81 |
| Glycerol montanate | 78 | 80 | 66 | 71 |
| Pentaerythritol montanate | 78 | 80 | 66 | 70 |
| Montanic acid complex ester** | 86 | 86 | 81 | 82 |

* ®Loxiol G70S from Cognis
** ®Licolub WE 40 from Clariant GmbH

TABLE 5
Release action

| | Transparency in % | | | |
|---|---|---|---|---|
| | (at test product content of) | | | |
| | 0.3% | | 0.5% | |
| Ester type | KZ | WZ | KZ | WZ |
| TMP mixed ester | 28 | 32 | 33 | 38 |
| Glycerol mixed ester | 24 | 35 | 27 | 35 |
| Pentaerythritol mixed ester | 32 | 35 | 36 | 38 |
| Pentaerythritol complex ester | 31 | 33 | 36 | 37 |
| of Example 4 | | | | |
| Glycerol monostearate | 29 | 34 | 36 | 38 |
| Fatty acid complex ester* | 19 | 33 | 30 | 35 |
| Glycerol montanate | 30 | 33 | 27 | 37 |
| Pentaerythritol montanate | 31 | 33 | 33 | 35 |
| Montanic acid complex ester** | 14 | 30 | 28 | 33 |

* ®Loxiol G70S from Cognis
** ®Licolub WE 40 from Clariant GmbH

1. An ester or a partial ester of a polyhydric alcohol (component A) and carboxylic acids, comprising at least one of a carboxylic acid radical or a radical of a carboxylic acid mixture having from 24 to 34 carbon atoms (component B) and at least one of a carboxylic acid radical or a radical of a carboxylic acid mixture having from 8 to 22 carbon atoms (component C).

2. The ester or partial ester as claimed in claim 1, wherein the polyhydric alcohol has from 3 to 12 hydroxy groups.

3. The ester or partial ester as claimed in claim 1, wherein the polyhydric alcohol is selected from the group consisting of trimethylolpropane, trimethylolethane, pentaerythritol, glycerol, diglycerol, polyglycerol, sorbitol, dipentaerythritol, ditrimethylolpropane, ethoxylated derivatives of trimethylolpropane, trimethylolethane, pentaerythritol, glycerol, diglycerol, polyglycerol, sorbitol, dipentaerythritol, ditrimethylolpropane, and mixtures thereof.

4. The ester or partial ester as claimed in claim 1, wherein the polyhydric alcohol is trimethylolpropane, glycerol, and/or pentaerythritol or mixtures thereof.

5. The ester or partial ester as claimed in claim 1, wherein the at least one of a carboxylic acid radical or a radical of a carboxylic acid mixture contains from 8 to 18 carbon atoms.

6. The ester or partial ester as claimed in claim 1, further comprising at least one of a dicarboxylic acid radical or the radical of a dicarboxylic acid mixture (component D).

7. The ester or partial ester as claimed in claim 6, wherein the at least one of a dicarboxylic acid radical or the radical of the dicarboxylic acid mixture is derived from adipic acid, dodecanedioic acid, or dimer fatty acid.

8. The ester or partial ester as claimed in claim 1 having an acid number of from 5 to 25.

9. The ester or partial ester as claimed in claim 1 having a hydroxy number of from 5 to 400.

10. The ester or partial ester as claimed in claim 1 having a hydroxy number of from 5 to 150.

11. A plastic processing aid comprising an ester or partial ester as claimed in claim 1.

12. A process of processing a plastic comprising the step of adding the plastic processing aid as claimed in claim 11 to the plastic during the processing of the plastic.

13. The process as claimed in claim 11, wherein the plastic is a polyvinyl chloride, polyester, polycarbonate, polyamide, or polypropylene.

14. A dispersing agent for a pigment comprising an ester or partial ester as claimed in claim 1.

15. A process for preparing a micronizate comprising the step of adding an ester or partial ester as claimed in claim 1 during the preparation of the micronizate.

16. The method as claimed in claim 12, wherein the plastic is a thermoplastic.

17. A plastic made in accordance with the process of claim 12.

18. A micronizate made in accordance with the process of claim 15.

19. A pigment comprising a dispersing agent, wherein the dispersing agent is an ester or partial ester as claimed in claim 1.
